(19) United States
(12) Patent Application Publication
(10) Pub. No.: US 2021/0100480 A1
(43) Pub. Date: Apr. 8, 2021

(54) APPARATUS AND METHOD FOR DETECTING POSTURE USING ARTIFICIAL INTELLIGENCE

(71) Applicant: LG ELECTRONICS INC., Seoul (KR)

(72) Inventors: Hyunoh KANG, Seoul (KR); Younjae LEE, Seoul (KR); Hyounggil YOON, Seoul (KR); Seonghyok KIM, Seoul (KR); Hyungyu PARK, Seoul (KR); Homin LEE, Seoul (KR)

(73) Assignee: LG ELECTRONICS INC., Seoul (KR)

(21) Appl. No.: 16/803,358

(22) Filed: Feb. 27, 2020

(30) Foreign Application Priority Data
Oct. 4, 2019 (KR) 10-2019-0123111

(51) Int. Cl. A61B 5/11; G06N 20/00

(52) U.S. Cl. CPC A61B 5/1116; A61B 5/1123; A61B 5/1118; G06N 20/00

(57) ABSTRACT

Disclosed are a posture detection device and a posture detection method that can identify a user and determine the posture of a user by using artificial intelligence technology. An operation method of an electronic device to which artificial intelligence technology is applied includes acquiring sensing data measured by each of a plurality of sensors, determining whether a posture of a user is changed on the basis of the sensing data, acquiring statistical sensing data by statistically processing the sensing data when it is determined that the posture is changed, and identifying the user and determining the posture of the user on the basis of the statistical sensing data. With the use of an artificial intelligence machine learning technology, it is possible to improve posture determination accuracy and user identification accuracy.

### CROSS REFERENCE TO RELATED APPLICATION

The present application claims priority to Korean Patent Application No. 10-2019-0123111, filed Oct. 4, 2019, the entire contents of which is incorporated herein for all purposes by this reference.

### BACKGROUND

AI refers to the field of researching artificial intelligence or methodologies that can use artificial intelligence, and machine learning refers to the field of researching methodologies that define and solve various problems that are dealt with in the field of artificial intelligence. Machine learning is sometimes defined as an algorithm that improves the performance of a task through a consistent experience.

This artificial intelligence technology continues to develop and has been being extensively applied to industries to enhance the efficiency of devices.

On the other hand, sleeping is one of the important factors in the physical and mental health of humans. That is, sleeping with proper posture can provide good effects such as recovery from fatigue, improvement in immunity, improvement in concentration on tasks, relieving of stress, reduction of inflammation, and recovery of muscles.

Accordingly, posture support devices, posture assisting devices, posture correcting devices, and posture analyzing devices have appeared to assist users with proper sleeping posture to enable effective sleeping. However, at present, most of such devices require various sensors to be worn by a user. This may cause inconvenience to the user in taking proper posture, resulting posing a problem that the use of such a device is troublesome. Accordingly, there is an increasing demand for a device capable of analyzing a user's posture without being worn on the user's body.

### SUMMARY

Various embodiments relate to a posture detection device and a posture detection method. More particularly, embodiments relate to a posture detection device and method using artificial intelligence technology for identifying a user and detecting a posture of a user.

As an example, in order to analyze the sleeping posture of a use, the posture of the user was observed with sensors arranged under the bed. However, conventional sensors have a high dependency on the user's sleeping posture, and when the user takes a specific posture, it is difficult to analyze the posture of the user. In addition, when conventional devices are used, many sensors are required for analysis of sleeping posture.

Various embodiments of the present disclosure provide a smart posture detection device and method to which artificial intelligence technology is applied so that posture analysis accuracy can be improved in a case where the posture of a user is analyzed in a non-contact manner.

In addition, various embodiments of the present disclosure provide a smart posture detection device and method capable of identifying a user using an artificial intelligence algorithm on the basis of tendency of user's sleeping postures.

In addition, various embodiments of the present disclosure provide a smart posture detection device and method for maintaining a comfortable sleeping state for a user by feeding back and controlling environmental conditions suitable for each user.

The technical problems to be solved by the present disclosure are not limited to the ones mentioned above, and other technical problems which are not mentioned can be clearly understood by those skilled in the art from the following description.

According to various embodiments of the present disclosure, an electronic device to which artificial intelligence technology is applied includes: a plurality of sensors; a sensing unit operatively connected to the plurality of sensors; and at least one processor operatively connected to the sensing unit, wherein the at least one processor acquires sensing data measured by each of the plurality of sensors via the sensing unit, determines whether a posture change is made on the basis of the sensing data, and acquires statistical sensing data by statistically processing the sensing data when it is determined that the posture change is made, and identifies a user and determines a posture of a user on the basis of the statistical sensing data.

According to various embodiments of the present disclosure, an operation method of an electronic device to which an artificial intelligence technology is applied includes acquiring sensing data measured by a plurality of sensors; determining whether a posture change of a user is made on the basis of the sensing data; acquiring statistical sensing data by statistically processing the sensing data when it is determined that the posture change; and identifying a user and determining a posture of a user on the basis of the statistical sensing data.

According to various embodiments of the present disclosure, the user identification accuracy and the posture determination accuracy can be improved by using an artificial intelligence machine learning technique in which data measured by sensors are used as an input.

In addition, according to various embodiments of the present disclosure, the processing speed can be increased by performing, at the same time, signal acquisition and processing, and posture determination and user identification.

In addition, according to various embodiments of the present disclosure, since the device is provided with a plurality of learning regions, a specific user can be accurately identified, and a specific posture can be accurately analyzed.

The effects and advantages that can be achieved by the present disclosure are not limited to the ones mentioned above, and other effects and advantages which are not mentioned above can be clearly understood by those skilled in the art from the following description.

### BRIEF DESCRIPTION OF THE DRAWINGS

FIG. 1 is a diagram illustrating an electronic device employing an artificial intelligence technology according to various embodiments.

FIG. 2 is a diagram illustrating an AI server 200 using an artificial intelligence technology according to various embodiments.

FIG. 3 is a diagram illustrating an AI system 1 according to various embodiments.

FIG. 4 is a diagram illustrating a sensing unit 140 of the electronic device 100 according to various embodiments.

FIG. 5 is a diagram illustrating an example of sensing data obtained when a posture change is made.

FIG. 6 is a diagram illustrating an example of a 2D image generated by a processor of the electronic device.

FIG. 7 is a diagram illustrating an example of posture information output from the processor of the electronic device.

FIG. 8 is a flowchart illustrating a method in which the electronic device 100 determines user information and posture information according to various embodiments.

Throughout the drawings, like elements may be denoted by like reference numerals.

### DETAILED DESCRIPTION OF THE EMBODIMENTS

Hereinafter, embodiments of the present disclosure will be described in detail with reference to the accompanying drawings, and the same or similar components will be denoted by the same reference numerals throughout the drawings, and redundant description thereof will be omitted. The terms “module” and “unit” are used to simply name components used in embodiments to be described below only for the purpose of ease of description and are not meant to have distinct meanings or roles by the names. In addition, in describing embodiments disclosed herein, when it is determined that the detailed description of the related known technology may obscure the gist of the embodiments disclosed herein, the detailed description thereof will be omitted. In addition, the accompanying drawings are only for ease of understanding the embodiments disclosed herein, and the technical spirit disclosed in the specification are not limited by the accompanying drawings. That is, all changes that can be made without departing from the spirit and scope of the present disclosure, equivalents, and substitutions to the embodiments fall within the scope of the present invention.

Terms such as a first term and a second term may be used for explaining various constitutive elements, but the constitutive elements should not be limited to these terms. These terms are used only for the purpose for distinguishing a constitutive element from another constitutive element.

It will be understood that when any element is referred to as being “connected” or “coupled” to another element, one element may be directly connected or coupled to the other element, or an intervening element may be present therebetween. In contrast, it should be understood that when an element is referred to as being “directly coupled” or “directly connected” to another element, there are no intervening elements present.

AI refers to the field of researching artificial intelligence or methodologies that can use artificial intelligence, and machine learning refers to the field of researching methodologies that define and solve various problems that are dealt with in the field of artificial intelligence. Machine learning is sometimes defined as an algorithm that improves the performance of a task through a consistent experience.

An artificial neural network (ANN) is a model used in machine learning and may refer to an overall problem-solving model composed of artificial neurons (nodes) that forms a network through a combination of synapses. An artificial neural network may be defined with a connection pattern of neurons through different layers, a learning process of updating model parameters, and an activation function of generating an output value.

An artificial neural network may include an input layer, an output layer, and optionally one or more hidden layers. Each layer includes one or more neurons, and the artificial neural network may include synapses that connect neurons to neurons. In an artificial neural network, each neuron may output a function value of an active function for input signals, weights, and deflections that are input through synapses.

Model parameters refer to parameters determined through training and include weights of synaptic connections and deflections of neurons. On the other hand, a hyperparameter means a parameter whose value is set before the learning process begins in a machine learning algorithm. The hyperparameters include a learning rate, the number of repetitions, a mini batch size, an initialization function, and the like.

The goal of artificial neural network learning is to determine model parameters that can minimize a loss function. The loss function may be used as an index for determining an optimal model parameter in the learning process of an artificial neural network.

Machine learning can be categorized into supervised learning, unsupervised learning, and reinforcement learning.

Supervised learning refers to a method of training artificial neural networks with a given label for training data, and a label indicates a correct answer (or result value) that the artificial neural network should infer when the training data is input to the artificial neural network. Unsupervised learning may refer to a method of training artificial neural networks without a label for training data. Reinforcement learning may refer to a learning method that allows an agent defined in a certain environment to choose an action or a sequence of actions that maximizes cumulative reward in each state.

Among the artificial neural networks, machine learning implemented with a deep neural network (DNN) including a plurality of hidden layers is called deep learning. That is, deep learning is part of machine learning. Hereinafter, the term “machine learning” may refer to deep learning.

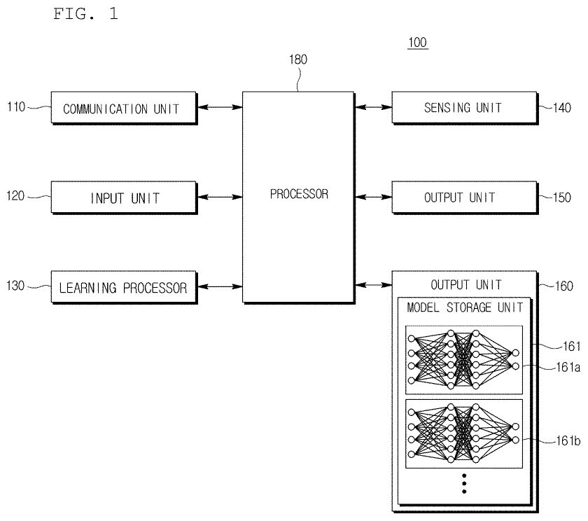

FIG. 1 is a diagram illustrating an electronic apparatus 100 employing an artificial intelligence technology according to various embodiments.

The electronic device 100 may be a stationary device or a mobile device. Examples of the electronic device 100 include a television (TV) set, a projector, a mobile phone, a smartphone, a desktop computer, a laptop computer, a digital broadcasting terminal, a personal digital assistant (PDA), a portable multimedia player (PMP), a navigation device, a tablet computer, a wearable device, or a set-top box (STB), s digital multimedia broadcasting (DMB) receiver, a radio, a washing machine, a refrigerator, a digital signage, robot, and a vehicle. The electronic device 100 employing artificial intelligence technology is also referred to as an artificial intelligence (AI) device.

Referring to FIG. 1, the electronic device 100 employing artificial intelligence technology may include a communication unit 110, an input unit 120, a learning processor 130, a sensing unit 140, an output unit 150, a memory unit 160, and a processor 180.

The communication unit 110 can communicate data with external devices such as an AI server or another AI device (i.e., another electronic device employing artificial intelligence functions) using wired and/or wireless communication technology. For example, the communication unit 110 may communicate sensor information, user inputs, trained models, control signals, and the like with external devices.

The communication unit 110 may use wireless communication technologies including global system for mobile communication (GSM), a code division multi-access (CDMA), long term evolution (LTE), 5G, wireless LAN (WLAN), wireless-fidelity (Wi-Fi), Bluetooth™, radio frequency identification (RFID), infrared data association (IrDA), and ZigBee, near field communication (NFC) or wired communication technologies including local area network (LAN), wide area network (WAN), metropolitan area network (MAN) and Ethernet.

The input unit 120 may acquire various types of data. The input unit 120 may include a camera for making an input of an image signal, a microphone for receiving an audio signal, and a user input unit for receiving information from a user. Here, the camera or microphone may be considered a kind of sensor, and the signal obtained from the camera or microphone may be considered sensing data or sensor information. Therefore, the camera or microphone may be included in the sensing unit 140.

The input unit 120 may acquire input data to be used when acquiring an output using training data and a training model for model training. The input unit 120 may acquire raw input data. In this case, the processor 180 or the learning processor 130 may extract input features as preprocessing on the input data.

The learning processor 130 may train models 161a and 161b, each being composed of artificial neural networks, with the training data. According to an embodiment of the present disclosure, the learning processor 130 may train the models 161a and 161b composed of a plurality of artificial neural networks. In this case, the training data for each model may be different according to the purpose of each model. Here, the trained artificial neural network may be referred to as a trained model. The trained model can be implemented in hardware, software or a combination of hardware and software. The trained model may be used to infer result values for new input data other than the training data, and the inferred result values may be used as a basis for determination to perform a specific operation. According to an embodiment of the present disclosure, the learning processor 130 may perform artificial intelligence processing in conjunction with a learning processor 240 of the AI server 200.

According to various embodiments of the present disclosure, the learning processor 130 may be integrated with the processor 180 of the electronic device 100. In addition, the trained model executed in the learning processor 130 may be implemented in hardware, software, or a combination of hardware and software. When the trained model is implemented partially or entirely in software, one or more instructions constituting the trained model may be stored in the memory unit 160, an external memory unit directly connected with the electronic device 100, or a memory unit built in an external device. The learning processor 130 may implement an AI processing program by reading the instructions from the memory unit and executing the instructions.

The sensing unit 140 may acquire at least one type of information among internal information of the electronic device 100, surrounding environment information of the electronic device 100, and user information, with the use of various sensors.

In this case, the sensing unit 140 may include a proximity sensor, an illumination sensor, an acceleration sensor, a magnetic sensor, a gyro sensor, an inertial sensor, an RGB sensor, an IR sensor, a fingerprint sensor, an ultrasonic sensor, an optical sensor, a microphone, a camera, a lidar, a radar, a pressure sensor, a force sensor, and the like.

The output unit 150 may generate outputs related to senses such as seeing, hearing, or touching. The output unit 150 may include a display unit for outputting visual information, a speaker for outputting auditory information, a haptic module for outputting tactile information, and the like.

The memory unit 160 may store data on the basis of which various functions of the electronic device 100 can be implemented. For example, the memory unit 160 may store input data acquired through the input unit 120, training data, trained models, learning history, instructions to be executed by the learning processor 130, instructions to be executed by the processor 180, and models (or artificial neural networks) that are already trained or which are being trained by the learning processor 130.

The processor 180 may determine at least one executable operation of the electronic device 100 on the basis of the information determined or generated by a data analysis algorithm or a machine learning algorithm. In addition, the processor 180 may execute the determined operation by controlling the components of the electronic device 100. Programs to be used by the processor 180 may be stored in the memory unit 160.

The processor 180 may request, retrieve, receive, or utilize data stored in the learning processor 130 or the memory unit 160, and control the components of the electronic device 100 such that as predicted operation or a desirable operation among at least one executable operation can be executed.

When an association with an external device is required to perform the determined operation, the processor 180 may generate a control signal to control the external device and transmit the generated control signal to the external device.

The processor 180 may obtain intention information in connection with the user input and determine the requirement of the user on the basis of the obtained intention information.

In an embodiment, the processor 180 may acquire the intention information corresponding to the user input by using at least one of a speech to text (STT) engine for converting a voice input into a character string or a natural language processing (NLP) engine for acquiring intention information of a natural language. At least a part of at least either one of the STT engine and the NLP engine may be composed of artificial neural networks trained according to a machine learning algorithm. At least one of the STT engine and the NLP engine may be an engine trained by the learning processor 130, by the learning processor 240 of the AI server 200, or by distributed processing of those.

The processor 180 collects history information including the details of the operation of the electronic device 100 or user feedback about the operation. Next, the processor 180 stores the collected history information in the memory unit 160 or the learning processor 130 or transmits the collected history information to an external device such as the AI server 200. The collected history information may be used to update the trained model.

The processor 180 may control at least part of the components of the electronic device 100 to execute an application program stored in the memory unit 160. In addition, the processor 180 may operate two or more components of the components included in the electronic device 100 in combination to execute the application program.

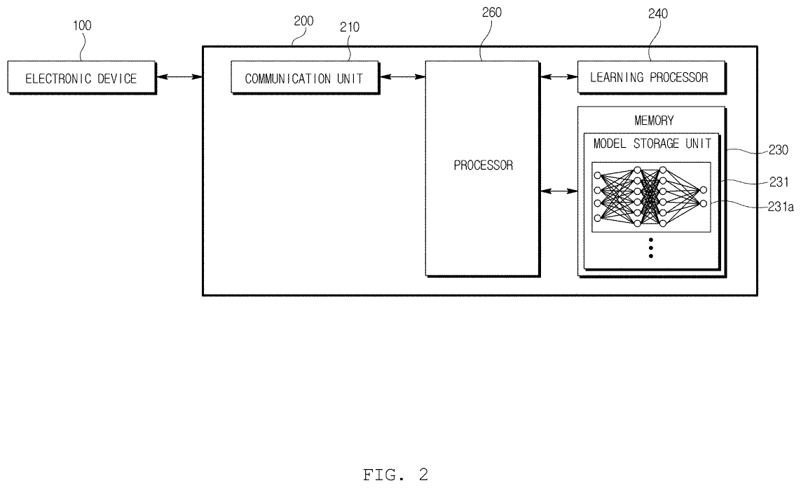

FIG. 2 is a diagram illustrating the AI server 200 using an artificial intelligence technology according to various embodiments.

Referring to FIG. 2, the AI server 200 may refer to a device for training an artificial neural network with the use of a machine learning algorithm or for using a trained artificial neural network. Here, the AI server 200 may be composed of a plurality of servers to perform distributed processing. The AI server 200 may be defined as a 5G network. According to an embodiment of the present disclosure, the AI server 200 may be configured as one component of the electronic device 100. According to another embodiment of the present disclosure, the AI server 200 may perform at least part of artificial intelligence processing in conjunction with the electronic device 100. For example, when the computing power of the electronic device 100 is insufficient, the electronic device 100 may request the AI server 200 to perform at least a part of or all the processes for artificial intelligence processing.

The AI server 200 may include a communication unit 210, a memory unit 230, a learning processor 240, and a processor 260.

The communication unit 210 may communication data with an external device such as the electronic device 100.

The memory unit 230 may include a model storage unit 231. The model storage unit 231 may store a model (or artificial neural network 231a) that is trained or is in a state of being trained by the learning processor 240.

The learning processor 240 may generate a trained model that is generated by training the artificial neural network 231a on the training data. The trained model may be implemented in the AI server 200 of the artificial neural network or may be implemented in an external device such as the electronic device 100 for use.

The trained model may be implemented in hardware, software or combination of hardware and software. When some or all the functions of the trained model are implemented in software, one or more instructions constituting the trained model may be stored in the memory unit 230.

The processor 260 may infer a result value with respect to new input data by using the trained model and generate a response or a control command on the basis of the inferred result value.

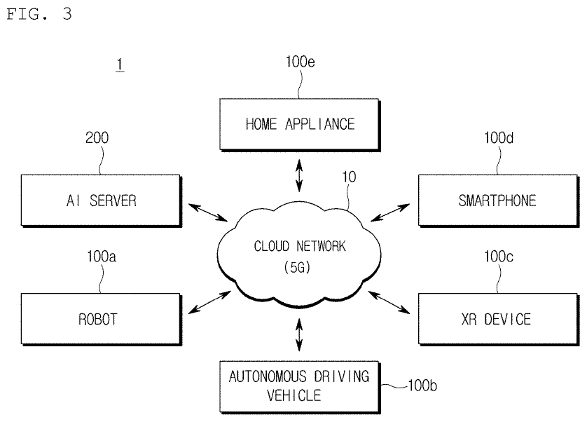

FIG. 3 is a diagram illustrating an AI system 1 according to various embodiments.

Referring to FIG. 3, the AI system 1 may be configured such that at least one device among an AI server 200, a robot 100a, an autonomous vehicle 100b, an XR device 100c, a smartphone 100d, and a home appliance 100e is connected to a cloud network 10. Here, the robot 100a, the autonomous vehicle 100b, the XR device 100c, the smartphone 100d, or the home appliance 100e to which an artificial intelligence technology is applied may be a specific example of the electronic device 100 employing the artificial intelligence technology of FIG. 1.

The cloud network 10 may constitute a portion of a cloud computing infrastructure or may refer to a network that is included in the cloud computing infrastructure. Here, the cloud network 10 may be configured with a 3G network, a 4G network (or long-term evolution (LTE) network), or a 5G network.

According to various embodiments, each of the electronic devices 100a to 100e and 200 constituting the AI system 1 may be connected to each other through the cloud network 10. According to one embodiment of the present disclosure, the electronic devices 100a to 100e and 200 may communicate with each other through a base station. Alternatively, the electronic devices 100a to 100e and 200 may directly communicate with each other without using a base station.

The AI server 200 may include a server that performs AI processing and a server that performs operations of big data.

The AI server 200 is connected, through the cloud network 10, to at least one of the robot 100a, the autonomous vehicle 100b, the XR device 100c, the smartphone 100d, and the home appliance 100e which are electronic devices, each employing artificial intelligence technology, thereby constituting the AI system 1. The AI server 200 may aid in performing the AI processing of the connected electronic devices 100a to 100e.

According to various embodiments of the present disclosure, the AI server 200 may train an artificial neural network according to a machine learning algorithm on behalf of the electronic devices 100a to 100e, and then store the trained model therein or transmit the trained model to the electronic devices 100a to 100e.

According to various embodiments of the present disclosure, the AI server 200 receives input data from the electronic devices 100a to 100e, infers a result value with respect to the received input data by using the trained model, generates a response or a control command based on the inferred result value, and transmits the response or the control command to the electronic devices 100a to 100e.

According to various embodiments of the present disclosure, the electronic devices 100a to 100e may infer a result value with respect to the input data by using a direct trained model and generate a response or a control command based on the inferred result value.

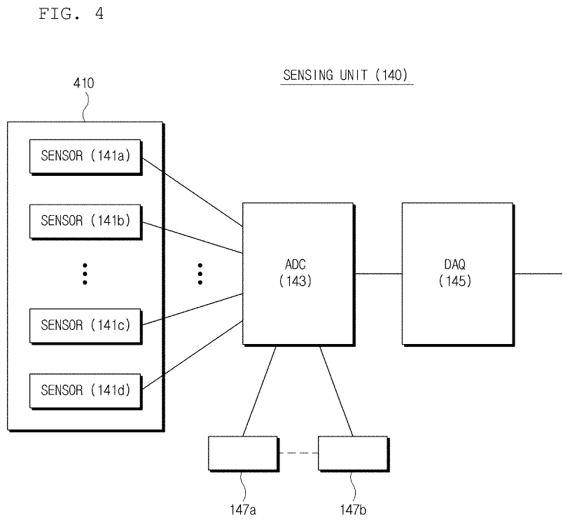

FIG. 4 is a diagram illustrating a sensing unit 140 of the electronic device 100 according to various embodiments.

Referring to FIG. 4, the sensing unit 140 may include a plurality of sensors 141a, 141b, 141c, and 141d, an analog-to-digital converter (ADC) 143, and a data acquisition unit (DAQ) 145. In addition, the sensing unit 140 may further include sensors 147a and 147b for measuring the surrounding environment parameters. According to one embodiment, the ADC 143 and the DAQ 145 may be implemented as one chip such as a system-on-chip (SOC), an application specific integrated circuit (ASIC), or a field programmable gate array (FPGA).

According to various embodiments of the present disclosure, the plurality of sensors 141a, 141b, 141c, and 141d may be combined with a bed mattress 410 and may be distributed through the entire area of the mattress 410 to determine a posture of the user. In one embodiment, the plurality of sensors 141a, 141b, 141c, and 141d may be arranged through the bed mattress 410 at regular intervals.

As the plurality of sensors 141a, 141b, 141c, and 141d, any types of sensors capable of detecting a force or pressure applied to them may be used. Each of the plurality of sensors 141a, 141b, 141c, and 141d may generate an analog signal proportional to the magnitude of the force or pressure applied thereto. For example, each of the sensors 141a, 141b, 141c, and 141d may be an electrostatic sensor, a force sensor, or a pressure sensor. The number of the sensors 141a, 141b, 141c, and 141d may range from four to eight. In addition, according to an exemplary embodiment, each of the sensors 141a, 141b, 141c, and 141d may output a voltage in a range from 0 V to 5 V or information corresponding to the magnitude of the force or pressure applied thereto.

According to various embodiments, the ADC 143 may convert an analog signal into a digital signal. According to an embodiment, the ADC 143 may detect a voltage signal ranging from 0 V and 5 V measured each of the plurality of sensors 141a, 141b, 141c, and 141d, and convert the voltage signal into a digital signal corresponding to each voltage signal. The digital signal may be a signal composed of a plurality of bits each having a value of 0 or 1. According to an embodiment, the digital signal may be configured with 8 bits or 16 bits, and the resolution may vary according to the number of bits.

According to various embodiments of the present disclosure, besides the plurality of sensors 141a, 141b, 141c, and 141d, the ADC 143 also may be connected with sensors 147a and 147b such as a breathing sensor, a temperature sensor for measuring the temperature of a mattress 410, an ambient temperature sensor, a humidity sensor, an illuminance sensor, and a noise sensor, thereby detecting a user's surrounding environment, for example, a sleeping environment during sleep. The ADC 143 may convert an analog signal output from each of those sensors into a digital signal. According to another embodiment, some sensors may output a digital signal instead of an analog signal. In the case of the sensors outputting a digital signal, the output digital signals may be directly input to the DAQ 145 or the processor 180 without undergoing signal conversion.

According to various embodiments of the present disclosure, the DAQ 145 may acquire sensing data from the digital signals output from the ADC 143. According to an embodiment, the DAQ 145 may acquire a signal for each sensor every first time period (for example, every 30 ms). According to another exemplary embodiment, the ADC 143 converts an analog signal input from each sensor into a digital signal every first time period (for example, every 30 ms) and outputs the digital signal, and the DAQ 145 outputs the digital signal output from the ADC 143.

In addition, the DAQ 145 may transfer the sensing data acquired in a first period for each sensor to the processor 180.

According to various embodiments of the present disclosure, the learning processor 130 acquires sensing data of each of the plurality of sensors 141a, 141b, 141c, and 141d from the DAQ 145 of the sensing unit 140 or the processor 180, statistically processes the sensing data to obtain the processed data (hereinafter referred to as statistical sensing data), and uses the statistical sensing data as training data to train the models 161a and 161b, each being composed of artificial neural networks.

According to various embodiments of the present disclosure, the learning processor 130 may generate at least two trained models. One trained model is a model for determining a user's posture. The user's posture may be one posture selected from among front, side, side crouched, back, and sitting. The remaining trained model may be a model for identifying a user and may determine who is currently sleeping on a mattress 410.

According to various embodiments of the present disclosure, the force or pressure applied to each sensor of the plurality of sensors 141a, 141b, 141c, and 141d varies according to who is the user or the posture of the user. By comparing, analyzing, or combining the magnitudes of the forces or pressures applied to the plurality of sensors 141a, 141b, 141c, and 141d, it is possible to identify a user and/or determine a posture of a user.

According to various embodiments of the present disclosure, the learning processor 130 may train a model on the basis of the input result data of each sensor. According to an embodiment, an artificial neural network model for determining the posture of a user may be trained according to a supervised learning method. When a user is positioned in a specific posture on a mattress 410, sensing data is obtained by each of the plurality of sensors 141a, 141b, 141c, and 141d, statistical processing is performed on the sensing data to produce the statistical sensing data, the statistical sensing data is set as training data to be input to the model, and the model is trained according to a supervised learning method by using posture information as a label. Furthermore, according to another embodiment, an artificial neural network model may be trained for identification of user. When a specific user is positioned on a mattress 410, sensing data is obtained by each of the plurality of sensors 141a, 141b, 141c, and 141d, statistical processing is performed on the sensing data to produce the statistical sensing data, the statistical sensing data is set as training data to be input to the model, and the model is trained according to a supervised learning method by using the specific user as a label. In this case, a series of statistical sensing data may be a sleeping pattern indicating a change in posture of the specific user during sleep. When the models are trained according to the supervised learning method, the user and the posture of the user can be identified. According to another embodiment, statistical processing may be performed on sensing data measured by each of the plurality of sensors 141a, 141b, 141c, and 141d for each of various users and for each of various postures of each of the users to obtain statistical sensing data. The models are trained according to an unsupervised learning method in which the obtained statistical sensing data is input to artificial neural network models as training data without labels. In the case of being trained according to an unsupervised learning method, classification of users and postures is possible, but it is difficult to specify users and postures.

Table 1 shows examples of training data for supervised learning. Each of the values in Table 1 is statistical sensing data obtained by collecting data sensed by each sensor in a second period and by statistically processing the collected data.

TABLE 1

| | Sensor | Sensor | Sensor | Sensor | Sensor | Sensor | Sensor | Sensor |
|---|---|---|---|---|---|---|---|---|
| Label | 1 | 2 | 3 | 4 | 5 | 6 | 7 | 8 |
| User A front | 2.02762 | 2.07697 | 2.00996 | 1.92085 | 2.65916 | 2.50165 | 2.38286 | 2.50746 |
| User A side | 2.09568 | 2.08047 | 2.04176 | 1.96335 | 2.73220 | 2.58187 | 2.52544 | 2.50760 |
| User A | 2.22454 | 2.11076 | 2.0863 | 1.95631 | 2.45850 | 2.62603 | 2.49599 | 2.48390 |
| Side crouched | | | | | | | | |
| User A back | 2.13536 | 2.04010 | 1.96960 | 1.92267 | 2.70623 | 2.54563 | 2.50869 | 2.52179 |
| User A sit | 2.25679 | 2.21337 | 2.06356 | 1.91357 | 2.52929 | 2.43617 | 2.55570 | 2.57326 |

According to various embodiments of the present disclosure, the processor 180 can identify a user or determine a posture of a user on the basis of the sensing data input from the sensing unit 140 by using a trained model that is generated through training by the learning processor 130. According to one embodiment, the processor 180 inputs the statistical sensing data, which is obtained by statistically processing the sensing data input from the sensing unit for each of the sensors, to a trained model generated by the learning processor 130, acquires a result from the trained model, and identifies a user and/or determines a posture of a user.

According to various embodiments, the processor 180 does not continuously generate the statistical sensing data to be input to the trained model. That is, the processor 180 performs statistical processing on the sensing data of each of the sensors to generate the statistical sensing data and inputs the statistical sensing data to the trained model only when it is determined that the posture of a user is changed. According to one embodiment, the processor 180 may generate the statistical sensing data only when a change in the value of the sensing data of each of at least a portion (for example, 50%) of the sensors mounted on a mattress 410 is greater than a predetermined threshold value (for example, 1 V). For example, when the number of the sensors mounted on the mattress 410 is eight in total and a change in the value of the sensing data of each of four or more sensors is equal to or greater than 1 V, the processor 180 may generate the statistical sensing data for each sensor. In addition, when the number of the sensors mounted on the mattress 410 is 8 in total and a change in the value of the sensing data of each of two or more sensors is equal to or greater than 1.5 V, the processor 180 may generate the statistical sensing data for each of the sensors.

The processor 180 may not generate the statistical sensing data during a period in which the sensing data considerably fluctuates and may generate the statistical sensing data when the sensing data is stabilized. For example, when the user changes its posture from a first posture to a second posture, that is, when the body of the user moves, the force or pressure applied to each of the sensors is highly likely to sharply change. When the second posture of the user is maintained, the force or pressure applied to each of the sensors is not likely to change but is kept stable. Therefore, the sensing data is stabilized.

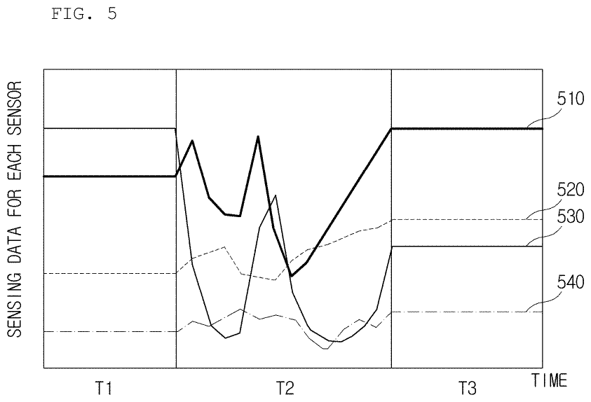

FIG. 5 is a diagram illustrating an example of sensing data obtained when a posture change is made.

Referring to FIG. 5, the values of sensing data items 510, 520, 530, and 540 do not fluctuate for a period T1. Thus, the period T1 is referred to as a stabilized period. However, in a period T2 during which the posture of the user is being changed, the values of the sensing data items 510, 520, 530, and 540 considerably fluctuate. After the posture change of the user is completed (period T3), the values of the sensing data items 510, 520, 530, and 540 do not fluctuate. The period T2 during which the posture of the user is being changed is referred to as a transition period.

The processor 180 may generate the statistical sensing data when the transition period switches to the stabilized period, that is, when the sensing data become stable, as illustrated in FIG. 5. According to one embodiment, a change in the value of the sensing data for each of the sensors is 1% or less, the period is determined as the stabilized period and the statistical sensing data is generated.

In a case where the stabilized period is reached after the posture of the user is changed, the processor 180 generates first statistical sensing data. When the stabilized state is maintained, since the value of the sensing data for each of the sensors is not likely to significantly change, additional statistical sensing data is not generated until the next posture is made.

The processor 180 can identify a specific user or determine a posture of a user on the basis of the statistical sensing data. In order to identify a specific user or determine a posture of a user, the processor 180 may use a trained model generated by the learning processor 130. According to one embodiment, a trained model for determining a posture of a user and a trained model for identifying a specific user are both used. The trained model for determining a posture of a user may differ from the trained model for identifying a specific user. According to an embodiment, the trained model for determining a posture of a user receives a piece of statistical sensing data as input data and provides a posture corresponding to the input piece of the statistical sensing data on the basis of the result of the learning. According to another embodiment, the trained model for identifying a specific user receives a plurality of pieces of statistical sensing data as input data and provides user information corresponding to the plurality of pieces of statistical sensing data on the basis of the result of the learning. The statistical sensing data input to the trained models may be a value obtained from the sensing data that is stably maintained and measured in a stabilized period (for example, the period T1 or T3 in FIG. 5). In the case of the transition period (for example, the period T2 in FIG. 5) during which the value of the sensing data significantly changes, the statistical sensing data is generated and thus no statistical sensing data is input to the trained models during this period. Therefore, the processor 180 can identify a specific user and determine a posture of a user by using a machine learning algorithm that uses a trained model generated by the learning processor 130. The posture of a user may be any posture selected from among front, side, side crouched, back, and sitting.

The processor 180 may store statistical sensing data that varies with time and store users and postures associated with the statistical sensing data. The processor 180 may generate a two-dimensional image that can be visually checked by the user on the basis of the stored data.

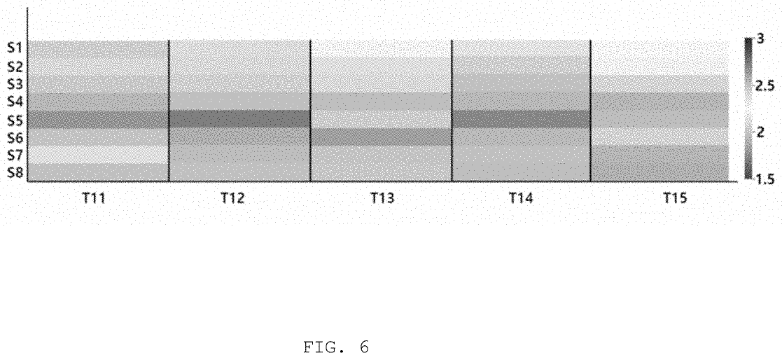

FIG. 6 is an example of the two-dimensional image generated by the processor 180 of the electronic device.

Referring to FIG. 6, the processor 180 generates a two-dimensional image in which different colors or different grayscales appear based on the magnitude of pressure or force measured by each of sensors S1 to S8 for each of time periods T11 to T15.

In addition, the processor 180 may construct a database based on the generated two-dimensional image in a cloud server so that the user can check his or her life pattern.

In addition, the processor 180 may determine a sleep quality of a user by analyzing a sleeping posture and/or a sleeping environment. To this end, the processor 180 may obtain additional information such as the temperature of the mattress 410, ambient temperature, noise, and humidity by using additional sensors 147a and 147b. The processor 180 may determine how comfort the sleeping environment is and determine the sleep quality of the user on the basis of the additional information.

In addition, the processor 180 may output posture information through an output unit 150. The posture information may include statistical sensing data according to time and/or the determined postures of the user associated with the statistical sensing data. In addition, the processor 180 can output the user information of the identified user.

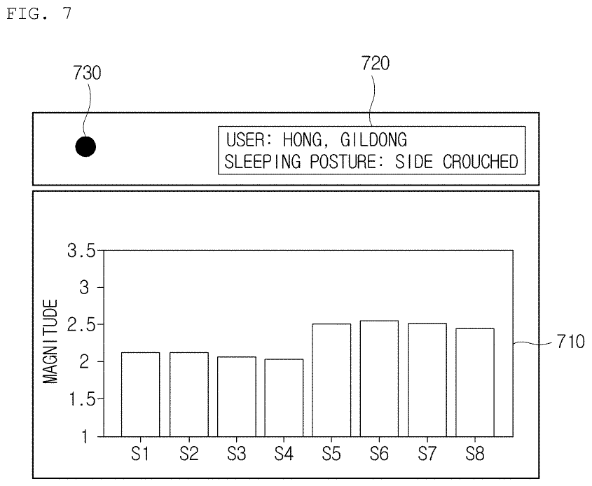

FIG. 7 is a diagram illustrating an example of posture information output from the processor of the electronic device.

The processor 180 may determine the sleeping posture of the user on the basis of the sensing data acquired through the plurality of sensors, and may display the determined sleeping posture to the user through the screen of the output unit 150 so that the user can check his or her sleeping posture. Referring to FIG. 7, the processor 180 may display a graph 710 indicating the magnitude of the force or pressure measured by each of the plurality of sensors, the determined user information, and the sleeping posture 720 of the user on the screen. According to an embodiment of the present disclosure, the processor 180 further includes an indicator 730 indicating whether the determination is in progress. For example, the indicator 730 flashes red in the state in which the determination is in progress and flashes green in the state in which the determination on the sleeping posture is finished.

According to various embodiments, an electronic device (for example, the electronic device 100 of FIG. 1) to which artificial intelligence technology is applied includes a plurality of sensors (for example, the sensors 141a, 141b, 141c, and 141d of FIG. 4), a sensing unit (for example, the sensing unit 140 of FIG. 1) operably connected to the plurality of sensors, and at least one processor (for example, the processor 180 of FIG. 1 and/or the learning processor 130 of FIG. 1) operably connected to the sensing unit. The at least one processor acquires sensing data measured by each of the plurality of sensors via the sensing unit, determines whether a posture change is made on the basis of the sensing data, statistically processes the sensing data to acquire statistical sensing data when it is determined that the posture change is made, and identifies a user or determines a posture of a user on the basis of the statistical sensing data.

According to various embodiments, the at least one processor may execute at least a portion of instructions of a first trained model to which artificial intelligence technology is applied to determine the posture of a user and at least a portion of instructions of a second trained model to which artificial intelligence technology is applied to identify a user, thereby determining the user and the posture of the user by using the statistical sensing data as input data for the first trained model and the second trained model.

According to various embodiments, the sensing unit may acquire the sensing data for each of the sensors at a first time interval the at least one processor may determine that the posture change is made when a difference between a value of the sensing data measured in a previous period and a value of the sensing data measured in a current period by each of at least a portion of the sensors is equal to or greater than first threshold value. According to one embodiment, the number of the at least a portion of the sensors may be half or more than half a total number of the plurality of sensors, and the first threshold value may be ⅕ times the maximum value that can be measured as the sensing data.

According to various embodiment, the at least one processor may collect the sensing data for a second time and calculate one value selected from among an average value, a mode value, and a median value of the collected sensing data, thereby obtaining the statistical sensing data for each of the sensors.

According to various embodiments, the at least one processor may determine a time period as a stabilized period or a transition period and acquire the statistical sensing data after the stabilized period is reached after it is determined that the posture change is made, wherein the stabilized period refers to a period in which a difference between a value of the sensing data measured in a previous period and a value of the sensing data measured in a current period is less than a second threshold value or a first threshold ratio, and the transition period refers to a period in which the difference between the value of the sensing data measured in the previous period and the value of the sensing data measured in the current period is equal to or greater than the second threshold value or a second threshold ratio.

According to various embodiments, the at least one processor may determine the posture of the user by inputting a piece of the statistical sensing data to the first trained model and identify the user by inputting a series of pieces of the statistical sensing data to the second trained model.

According to various embodiments, the electronic device may further include an output unit including a display unit and being operably connected to the at least one processor, and the at least one processor may the identified user, the determined posture of the user, and/or the statistical sensing data on the display unit.

According to various embodiments, the electronic device may further include a memory unit operably connected to the at least one processor, and the at least one processor may generate and store a two-dimensional image in the memory unit, wherein the two-dimensional image is configured such that an x axis represents passage of time, a y axis represents each of the plurality of sensors, and each point at x and y coordinates represents the statistical sensing data for a corresponding one of the plurality of sensors and wherein the statistical sensing data is expressed in colors or grayscales. The at least one processor may additionally store the two-dimensional image in a cloud server on a cloud network.

According to various embodiments, the electronic device may further include a communication unit operably connected to the at least one processor, wherein the at least one processor communicates with an external artificial intelligence server through the communication unit and executes at least a portion of functions of the first trained model and/or at least a portion of functions of the second trained model in conjunction with the artificial intelligence server.

According to various embodiments, the plurality of sensors may be sensors that can measure the magnitude of the force or pressure applied by the body of the user and may be distributed on a mattress on which a user can sleep.

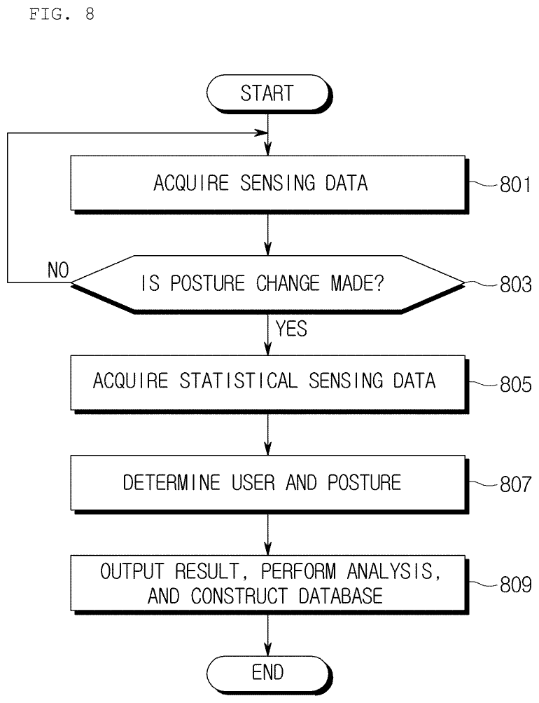

FIG. 8 is a flowchart illustrating a method in which the electronic device 100 determines user information and posture information according to various embodiments.

Referring to FIG. 8, in Step 801, the electronic device 100 acquires sensing data. The sensing data may be the magnitude of the force or pressure applied to each of the sensors by the body of the user. The sensing data is measured at a first time interval by each of the sensors (for example, the sensors 141a, 141b, 141c, and 141d of FIG. 4) distributed on a mattress.

According to various embodiments, in Step 803, the electronic device 100 determines whether the posture of the user is changed on the basis of the acquired sensing data. For example, when a change in the value of the sensing data of each of at least a portion (for example, 50%) of the plurality of sensors distributed on the mattress 410 is greater than a predetermined threshold value (for example, 1 V), it is determined that the posture of the user is changed. For example, when the number of the sensors provided on the mattress 410 is eight in total and a change in the value of the sensing data of each of four sensors of the eight sensors is 1 V or more, the electronic device 100 determines that the posture of the user is changed. Alternatively, when the number of sensors provided on the mattress 410 is eight in total and a change in the value of the sensing data of each of two sensors of the eight sensors is 1.5 V or more, the electronic device 100 determines that the posture of the user is changed. The criterion to determine whether the posture is changed may be stored in a memory unit.

According to various embodiments, in Step 805, the electronic device 100 acquires statistical sensing data. The statistical sensing data is obtained by collecting the sensing data for a second time and performing statistical processing on the collected sensing data. The statistical processing is to calculate an average value, a mode value, or a median value of the collected sensing data for each of the sensors. According to one embodiment, the electronic device 100 does not acquire the statistical sensing data for the transition periods and acquires the statistical sensing data only for the stabilized periods. For example, when the posture of the user is changed from a first posture to a second posture, the body of the user moves. At this time, the magnitude of the force or pressure applied to each sensor by the body of the user considerably changes. After the switching to the second posture is completed, a change in the magnitude of the force or pressure applied to each sensor is not likely to be small. Therefore, the electronic device 100 does not acquire the statistical sensing data from the sensing data measured during the period in which the sensing data considerably fluctuates until the stabilized period in which the values of the sensing data are stable is reached. When the stabilized period is reached, the electronic device 100 acquires the statistical sensing data while determining that a new posture (second posture) is maintained after the posture of the user is changed from the first posture to the second posture.

According to various embodiments, in Step 807, the electronic device 100 can identify a user and determine the sleeping posture of a user on the basis of the acquired statistical sensing data. According to one embodiment, the electronic device 100 can identify a user and determine the sleeping posture of a user by using a trained model obtained by training an artificial intelligence neural network. The electronic device 100 may have two trained models respectively for identifying a user and determining the sleeping posture of a user. The electronic device 100 can preliminarily train an artificial intelligence neural network model through supervised learning that provides a label and training data to the artificial intelligence neural network model. The label may include user information of training data that is currently input and information on the postures of users. The training data for the model for determination of a sleeping posture may include a label and the strength or intensity of the force measured by each sensor as shown in Table 1. In addition, the training data for the model for identifying the user may include information on a series of sleeping posture changes of a user for a predetermined time (for example, 1 minute or 2 minutes). Accordingly, the electronic device 100 may input the acquired statistical sensing data to a trained model for identifying a user and a trained model for determining a posture and may determine a user and a sleeping posture from the analysis results of each trained model. Here, the user's sleeping posture may be one of the front, the side, the side crouched, the back, and the sitting. In addition, since the user identification requires more pieces of statistical sensing data than the sleeping posture determination, the process of identifying a user takes a longer time than the process of determining the sleeping posture. Thus, the result of the identification of a user is produced a little bit later as compared to the result of the determination of the sleeping posture.

According to one embodiment, each of the plurality of sensors provided on the mattress 410 outputs sensing data at time intervals of 30 ms, and the electronic device 100 collects sensing data for one second for each of the plurality of sensors ad calculates a statistical value (i.e., statistical sensing data) of the collected sensing data for each of the plurality of sensors. When the electronic device 100 determines that the posture is changed, the electronic device 100 may determine the posture on the basis of the acquired statistical sensing data, and may identify the user on the basis of 10 user-posture changes or user-posture changes that are made for a predetermined time ranging from 1 minute to 2 minutes.

According to various embodiments, in Step 809, the electronic device 100 may output a result of the determination, perform an analysis, and construct a database. According to an embodiment, as illustrated in FIG. 7, the electronic device 100 may configure a screen to be shown to the user on the basis of the determined result and the collected statistical sensing data and provide the result to the user.

According to an embodiment, the electronic device 100 may generate a two-dimensional image as shown in FIG. 6, which may indicate a change in statistical sensing data of each sensor over time and may construct a database. According to an embodiment, the two-dimensional image may be configured such that the x-axis represents time, the y-axis represents each sensor, and the magnitude of the force or pressure measured by each sensor is displayed in colors or grayscales. By generating the two-dimensional image, it is possible to further analyze the sleep quality of the user by determining how often the user changes his posture during sleep and in which posture he takes during the sleep.

According to another embodiment, the electronic device 100 may obtain additional information such as the temperature of the mattress 410, ambient temperature, noise, and humidity using the sensors 147a and 147b. The additional information may be used by the electronic device 100 to determine how comfort the sleeping environment is or to determine sleep quality.

According to a further embodiment, the electronic device 100 may construct a database with user information including determined posture information, generated two-dimensional image information, and analyzed sleep quality information, in a cloud server on a cloud network illustrated in FIG. 3, thereby enabling the user to check his or her life pattern.

According to various embodiments, an operation method of an electronic device to which artificial intelligence technology is applied includes: an operation of acquiring sensing data measured by each of a plurality of sensors; an operation of determining whether a posture change of a user is made on the basis of the sensing data; an operation of acquiring statistical sensing data by statistically processing the sensing data when it is determined that the posture change is made; and an operation of identifying the user or determining a posture of a user on the basis of the statistical sensing data.

According to various embodiments, the operation of identifying the user or determining the posture of the user on the basis of the statistical sensing data may include: an operation of executing at least part of functions of a first trained model to which artificial intelligence technology is applied, in order to determine the posture of the user; an operation of executing at least part of functions of a second trained model to which artificial intelligence technology is applied, in order to identify the user; and an operation of using the statistical sensing data as input data for the first trained model and the second trained model.

According to various embodiments, the operation of acquiring the sensing data measured by each of the plurality of sensors may include an operation of acquiring the sensing data for each of the plurality of sensors at first time intervals. The operation of determining whether the posture change of the user is made on the basis of the sensing data may include an operation of determining that the posture change is made when a difference between a value of the sensing data, measured in a current period, of at least one sensor of the plurality of sensors and a value of the sensing data, measured in a previous period, of the at least one of the plurality of the sensors is equal to or greater than a first threshold value. According to one embodiment, the number of the at least one sensor of the plurality of sensors may be half or more than half the number of the plurality of sensors, and the first threshold value may be ⅕ times the maximum value that can be measured as the sensing data.

According to various embodiments, the operation of acquiring the statistical sensing data by statistically processing the sensing data may include: an operation of collecting the sensing data for a second time and an operation of calculating at least one value among an average value, a mode value, and a median value of the collected sensing data.

According to various embodiments, the method may further include: an operation of determining each time period as a stabilized period or a transition period. The stabilized period refers to a period in which a different between a value of the sensing data measured in a previous period and a value of the sensing data measured in a current period is less than a second threshold value or a first threshold ratio, and the transition period refers to a period in which the different between the value of the sensing data measured in the previous period and the value of the sensing data measured in the current period is greater than the second threshold value or a second threshold ratio. The operation of acquiring the statistical sensing data by statistically processing the sensing data may further include an operation of acquiring the statistical sensing data after the stabilized period is reached when it is determined that the posture change is made.

According to various embodiments, the operation of identifying the user or determining the posture of the user on the basis of the statistical sensing data may include an operation of determining the posture of the user by inputting one piece of the statistical sensing data to the first trained model and an operation of identifying the user by inputting a series of pieces of the statistical sensing data to the second trained model.

According to various embodiments, the method may further include an operation of displaying the identified user, the posture of the user, and/or the statistical sensing data on a display unit.

According to various embodiment, the method may further include an operation of generating a two-dimensional image and storing the two-dimensional image in a memory unit, in which the two-dimensional image is configured such that an x axis represents passage of time, a y axis represents the plurality of sensors, and each point at x and y coordinates represents the statistical sensing data for a corresponding one of the plurality of sensors, and in which the statistical sensing data is expressed in color or in grayscale. In addition, the method may further include an operation of storing the two-dimensional image in a cloud server on a cloud network.

According to various embodiments of the present disclosure, the method may further include an operation of communicating with an external artificial intelligence server and an operation of executing at least part of the functions of a first learning training model and/or at least part of the functions of a second trained model in conjunction with the artificial intelligence server.

As described above, the device and method proposed in the present disclosure can improve the posture determination accuracy and the user identification accuracy of sensors by using artificial machine learning technology. In addition, the device and method proposed in the present disclosure use a plurality of trained models to improve processing speed, thereby simultaneously performing detection of drowsy driving (i.e. determination of sleeping posture) and user identification.

In addition, the artificial machine learning technology proposed in the present disclosure can be easily implemented by integrating a Python machine learning algorithm and a LabVIEW code.

In addition, the above description relates to a configuration in which the posture of a user during sleep is determined by placing a plurality of sensors on a mattress. However, the device and method proposed in the present disclosure can be applied to a case where a user is sitting in a chair or sitting in the driver's seat of a vehicle. In this case, the device and method can be used to determine the posture of the user. Specifically, the device and method can be used to determine drowsy driving by determining the posture of the driver and performing detailed analysis of the posture of the driver.

What is claimed is:

1. An electronic device using artificial intelligence technology, the electronic device comprising:
a plurality of sensors;
a sensing unit operatively connected to the plurality of sensors; and
at least one processor operatively connected to the sensing unit,
wherein the at least one processor acquires sensing data measured by each of the plurality of sensors via the sensing unit, determines whether or not a posture of a user is changed on the basis of the sensing data, obtains statistical sensing data by statistically processing the sensing data when it is determined that the posture is changed, and identifies the user and determines a posture of the user on the basis of the statistical sensing data.

2. The electronic device according to claim 1, wherein the at least one processor executes at least a portion of instructions of a first trained model to which the artificial intelligence technology is applied to determine the posture of the user and at least a portion of instructions of a second trained model to which the artificial intelligence technology is applied to identify the user, and
wherein the at least one processor identifies the user and determines the posture of the user by using the statistical sensing data as input data for the first trained model and the second trained model.

3. The electronic device according to claim 2, wherein the sensing unit periodically acquires the sensing data from each of the plurality of sensors at a first time interval, and wherein the at least one processor determines that the posture is changed when a difference between a value of the sensing data measured in a current period and a value of the sensing unit measured in a previous period from each of at least a portion of the plurality of sensors is equal to or greater than a first threshold value.

4. The electronic device according to claim 3, wherein the number of the at least a portion of the plurality of sensors is equal to or more than half a total number of the plurality of sensors, and wherein the first threshold value is ⅕ times a maximum value that can be measured as the sensing data.

5. The electronic device according to claim 3, wherein the at least one processor collects the sensing data measured for a second time and obtains the statistical sensing data for each sensor by calculating one value among an average value, a mode value, and a median value of the collected sensing data.

6. The electronic device according to claim 5, wherein the at least one processor determines each time period as a stabilized period or a transition period and acquires the statistical sensing data when the stabilized period is reached after it is determined that the posture is changed, the stabilized period being a period during which a difference between a value of the sensing data measured in a previous period and a value of the sensing data measured in a current period is less than a second threshold value or a first threshold ratio, the transition period being a period during which the difference between the value of the sensing data measured in the previous period and the value of the sensing data measured in the current period is equal to or greater than the second threshold value or the first threshold ratio.

7. The electronic device according to claim 2, wherein the at least one processor determines the posture of the user by inputting one piece of the statistical sensing data into the first trained model, and the at least one processor identifies the user by inputting a series of pieces of the statistical sensing data into the second trained model.

8. The electronic device according to claim 2, further comprising an output unit operatively connected to the at least one processor and configured to include a display unit,
wherein the at least one processor displays at least one piece of information selected from among the identified user, the determined posture of the user, and the statistical sensing data on the display unit.

9. The electronic device according to claim 2, further comprising a memory unit operatively connected to the at least one processor,
wherein the at least one processor generates and stores a two-dimensional image in a memory unit, the two-dimensional image being configured such that an x axis represents passage of time, an y axis represents each of the plurality of sensors, and each point at x and y coordinates represents the statistical sensing data of a corresponding one of the plurality of sensors, the statistical sensing data being displayed in colors or in grayscales according to the values thereof.

10. The electronic device according to claim 2, wherein the electronic device further comprises a communication unit operatively connected to the at least one processor,
the at least one processor communicates with an external artificial intelligence server through the communication unit, and
the at least one processor performs at least a portion of functions of the first trained model and/or at least a portion of functions of the second trained model in conjunction with the artificial intelligence server.

11. An operation method of an electronic device to which artificial intelligence technology is applied, the method comprising:
acquiring sensing data measured by each of a plurality of sensors;
determining whether a posture of a user is changed on the basis of the sensing data;
acquiring statistical sensing data by statistically processing the sensing data when it is determined that the posture of the user is changed; and
identifying a user and determining a posture of the user on the basis of the statistical sensing data.

12. The method according to claim 11, wherein the identifying of the user and determining the posture of the user comprises:
executing at least one function of a first trained model to which artificial intelligence technology is applied to determine the posture of the user;
executing at least one function of a second trained model to which the artificial intelligence technology is applied to identify the user; and
using the statistical sensing data as input data for the first trained model and the second trained model.

13. The method according to claim 12, wherein the acquiring of the sensing data measured by each of the plurality of sensors comprises periodically acquiring the sensing data corresponding to each of the plurality of sensors at a first time interval, and
the determining of whether the posture is changed on the basis of the sensing data comprises determining that the posture is changed when a difference between a value of the sensing data measured in a current period and a value of the sensing data measured in a previous period, of each of at least a portion of the plurality of sensors is equal to or greater than a first threshold value.

14. The method according to claim 13, wherein the number of the at least a portion of the plurality of sensors is half or more than half a total number of the plurality of sensors, and the first threshold value is ⅕ times a maximum value that can be measured as the value of the sensing data.

15. The method according to claim 13, wherein the acquiring of the statistical sensing data by statistically processing the sensing data comprises:
collecting the sensing data for a second time; and
calculating one value among an average value, a mode value, and a median value of the collected sensing data, thereby acquiring the statistical sensing data for each of the plurality of sensors.

16. The method according to claim 15, further comprising:
determining each time period as a stabilized period or a transition period, the stabilized period being a period during which a difference between a value of the sensing data measured in a previous period and a value of the sensing data measured in a current period is less than a second threshold value or a first threshold ratio, the transition period being a period during which the difference between the value of the sensing data measured in the previous period and the value of the sensing data measured in the current period is equal to or greater than the second threshold value or the first threshold ratio,
wherein the acquiring of the statistical data by statistically processing the sensing data comprises acquiring the statistical sensing data when the stabilized period is reached when it is determined that the posture is changed.

17. The method according to claim 12, wherein the identifying of the user and determining of the posture of the user on the basis of the statistical sensing data comprise:
determining the posture of the user by inputting one piece of the statistical sensing data into the first trained model; and
identifying the user by inputting a series of pieces of the statistical sensing data into the second trained model.

18. The method according to claim 12, further comprising displaying the identified user, the posture of the identified user, and/or the statistical sensing data on a display unit.

19. The method according to claim 12, further comprising:
generating and storing in a memory unit a two-dimensional image in which an x axis represents passages of time, a y axis represents the plurality of sensors, and each point at x and y coordinates represents the statistical sensing data expressed in colors or in grayscales for each of the plurality of sensors.

20. The method according to claim 12, further comprising:
communicating with an external artificial intelligence server; and
performing at least one function of the first trained model and/or at least one function of the second trained model in conjunction with the artificial intelligence server.
